(19) United States
(12) Patent Application Publication
(10) Pub. No.: US 2023/0301882 A1
(43) Pub. Date: Sep. 28, 2023

(54) CALCIUM CARBONATE AND ZINC OXIDE-LOADED MICROCAPSULES AND USES THEREOF

(71) Applicant: PHYTO TECH CORP., Rancho Santa Margarita, CA (US)

(72) Inventor: Cuie YAN, San Pedro, CA (US)

(21) Appl. No.: 18/189,892

(22) Filed: Mar. 24, 2023

Related U.S. Application Data
(60) Provisional application No. 63/323,408, filed on Mar. 24, 2022.

(51) Int. Cl. A61K 8/27; A61K 9/48; A61K 9/50; A61K 8/73; A61K 8/11; A61Q 5/02; A61Q 11/00; A61Q 19/00; A23L 5/42; A23L 29/00; A23L 29/212; A23L 29/25; A23L 13/40; A23L 17/40; A23L 2/58

(52) U.S. Cl. CPC A61K 8/27; A61K 9/485; A61K 9/5036; A61K 8/732; A61K 8/11; A61K 8/73; A61Q 5/02; A61Q 11/00; A61Q 19/001; A23L 5/42; A23L 29/015; A23L 29/212; A23L 29/25; A23L 13/432; A23L 13/422; A23L 17/40; A23L 2/58

(57) ABSTRACT

This disclosure provides CaCO₃ and/or ZnO-loaded microcapsules for use as whitening agents in foods, beverages, supplements, pharmaceutic products, cosmetic products, and personal care products.

### BACKGROUND OF THE INVENTION

### Field of Invention

This disclosure provides a dry powder comprising calcium carbonate and/or zinc oxide (ZnO)-loaded microcapsules for use as a whitening agent in foods, beverages, supplements, pharmaceutic products, cosmetic products, and personal care products.

### Background

One of the most widely used food pigments is titanium dioxide (E171), an odorless powder that enhances the white color or opacity of foods. The most common foods containing titanium dioxide are chewing gum, candies, pastries, chocolates, coffee creamers, and cake decorations. Titanium dioxide is also present in cosmetics, paints, and medicines.

In recent decades, concerns about the risks of titanium dioxide consumption have grown. The Food and Drug Administration (FDA) categorizes titanium dioxide as Generally Recognized as Safe, but other organizations have issued warnings. For example, the European Food Safety Authority (EFSA) has concluded that titanium oxide should not be considered safe as a food additive due to uncertainties about possible inflammation and neurotoxicity. There exists a need for safe alternatives to titanium dioxide for use as whitening agents in consumer products.

### BRIEF SUMMARY OF THE INVENTION

In one aspect, the disclosure provides microcapsules for use as a whitening agent comprising an inner core and a primary shell surrounding the inner core, wherein (i) the inner core comprises food grade calcium carbonate (CaCO₃) and/or zinc oxide (ZnO); and (ii) the primary shell comprises food grade modified starch or gum Arabic.

The microcapsules described above are referred to as “CaCO₃ and/or ZnO-loaded microcapsules.”

In another aspect, CaCO₃ and/or ZnO-loaded microcapsules are spray dried and the resulting dry powder is used as a whitening agent in foods and pharmaceutical products. For example, the dry powder comprising CaCO₃ and/or zinc oxide (ZnO)-loaded microcapsules can be used to bring a bright and smooth white appearance to pastries or confectionary, or to opacify yogurt or ice cream. The dry powder comprising CaCO₃ and/or zinc oxide (ZnO)-loaded microcapsules can also be used as a whitening agent in candy, chewing gum, bakery products, cake icing, and meat analogs. The dry powder comprising CaCO₃ and/or zinc oxide (ZnO)-loaded microcapsules can also be used as a whitening agent in beverages, e.g., soy milk, coffee, and coffee creamers. The dry powder comprising CaCO₃ and/or zinc oxide (ZnO)-loaded microcapsules can also be used as a whitening agent in pharmaceutical products, e.g., in tablet, chewing gum, thin film, or capsule preparations.

### BRIEF DESCRIPTION OF THE DRAWINGS

FIG. 1 is an image showing the whitening affect of BC WA 4 as compared to TiO₂ in a 6% gelatin suspension at neutral pH.

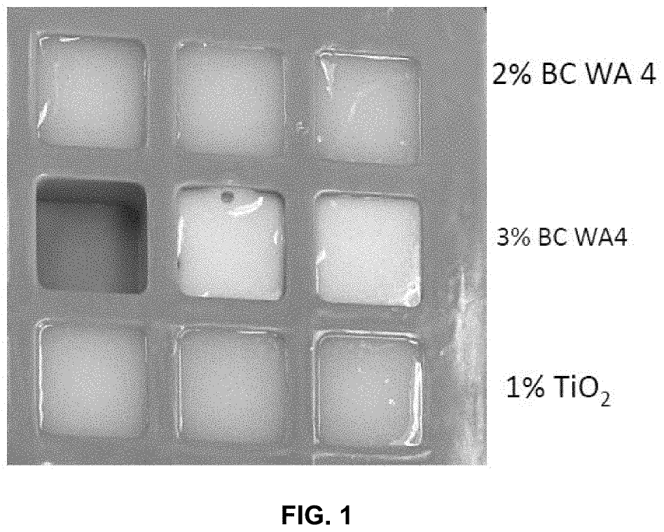

FIG. 2 is an image showing the whitening affect of BC WA 4 as compared to TiO₂ in a 6% gelatin suspension at pH 3.

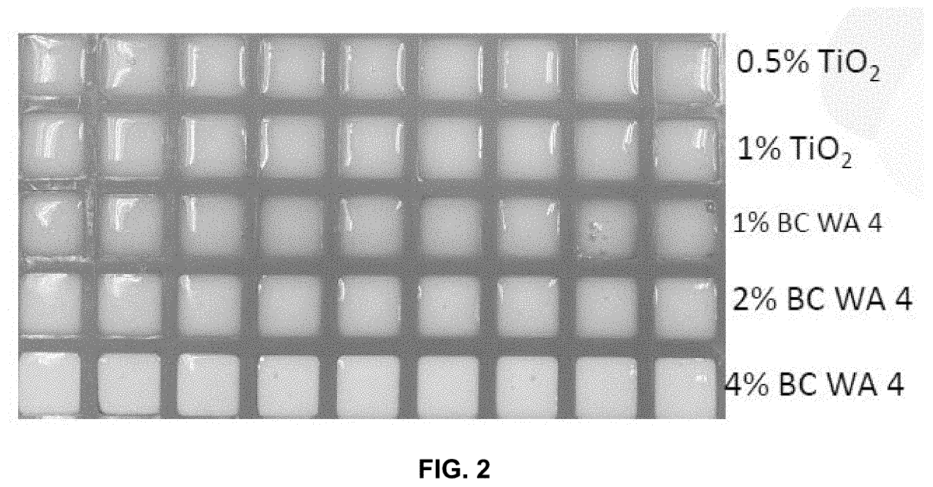

FIG. 3 is an image showing the whitening affect of BC WA 4 as compared to TiO₂ in 1% konjac gel at pH 4.5.

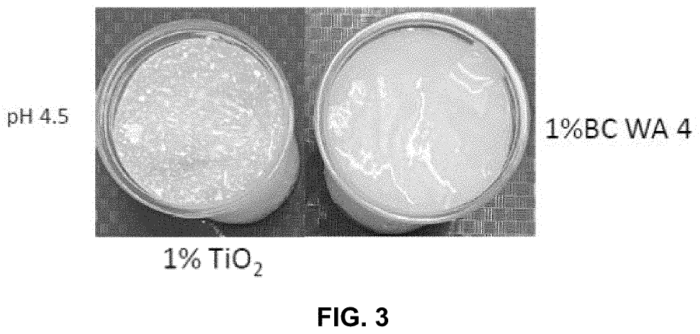

FIG. 4 is an image showing the whitening affect of BC WA 4 as compared to TiO₂ in 1% konjac gel at pH 5.7.

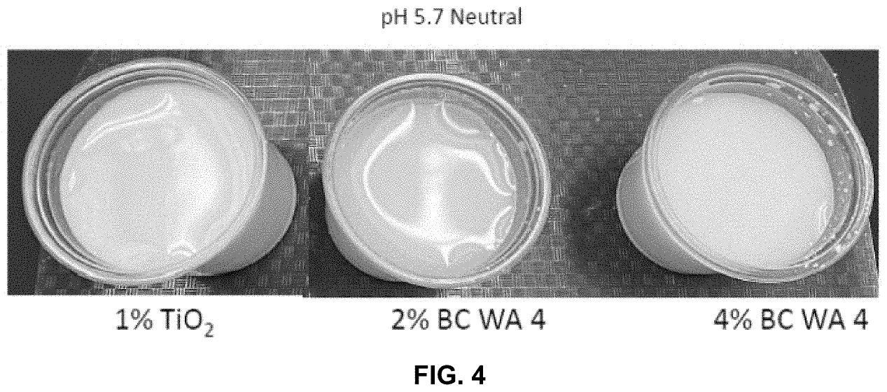

FIG. 5 is an image showing the whitening affect of BC WA 4 as compared to TiO₂ in coffee creamer.

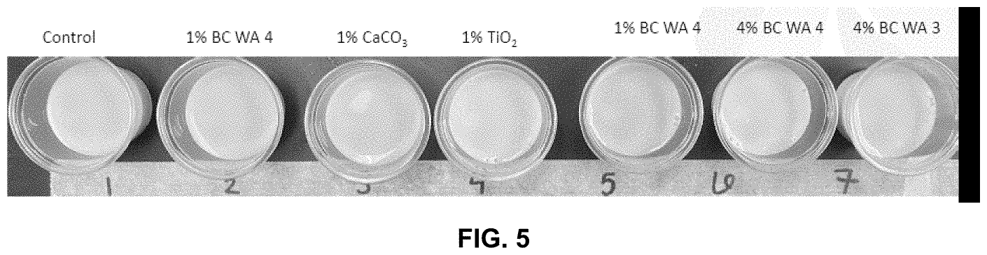

FIG. 6 is an image showing the whitening affect of BC WA 4 as compared to TiO₂ in yogurt.

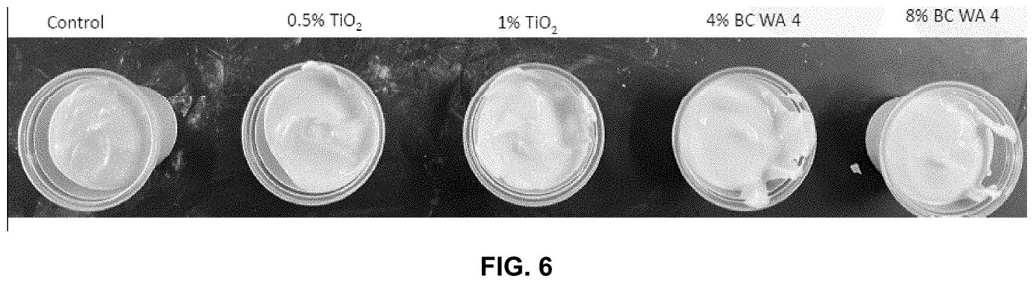

FIG. 7 is an image showing the whitening affect of BC WA 4 as compared to TiO₂ in a plant-based meat replacement.

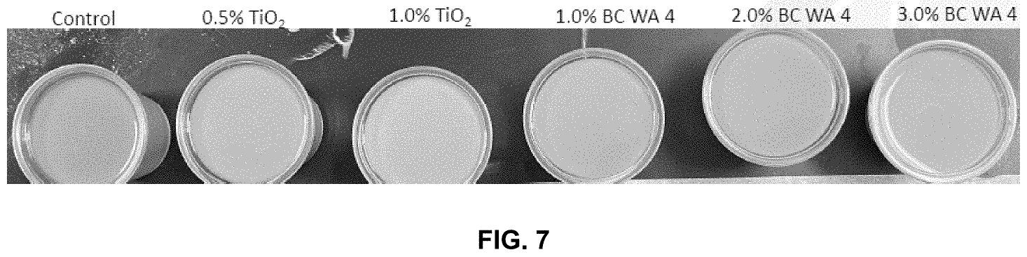

FIG. 8 is an image showing the whitening affect of BC WA 4 as compared to TiO₂ in cupcakes.

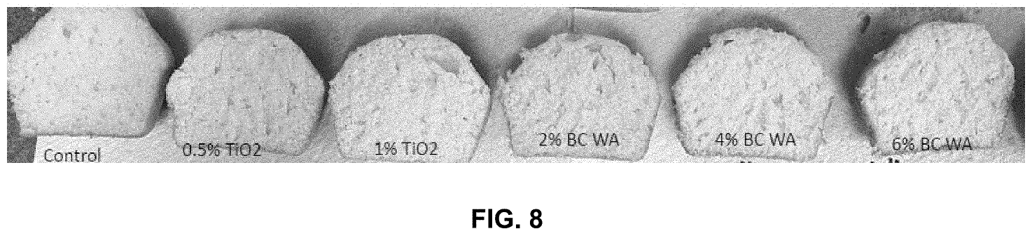

FIG. 9 is an image showing the whitening affect of BC WA 4 as compared to TiO₂ in icing.

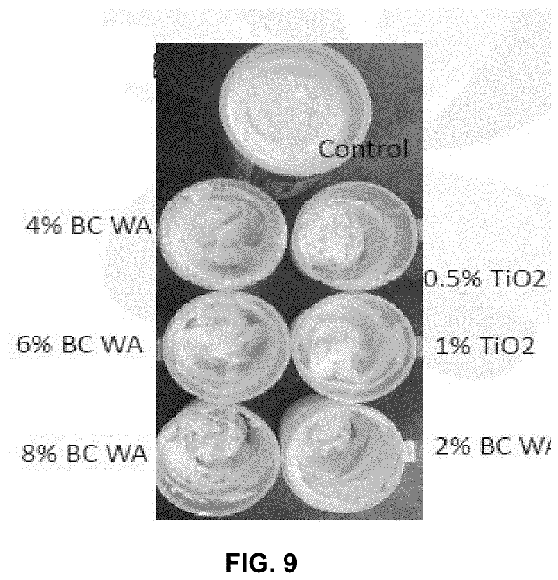

FIG. 10 is an image showing the whitening affect of BC WA 4 as compared to TiO₂ in lip balm.

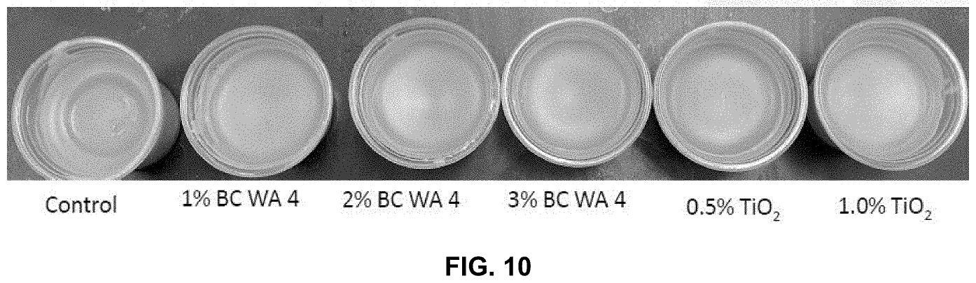

FIG. 11 is an image showing the whitening affect of BC WA 4 as compared to TiO₂ in cosmetic eye shadow.

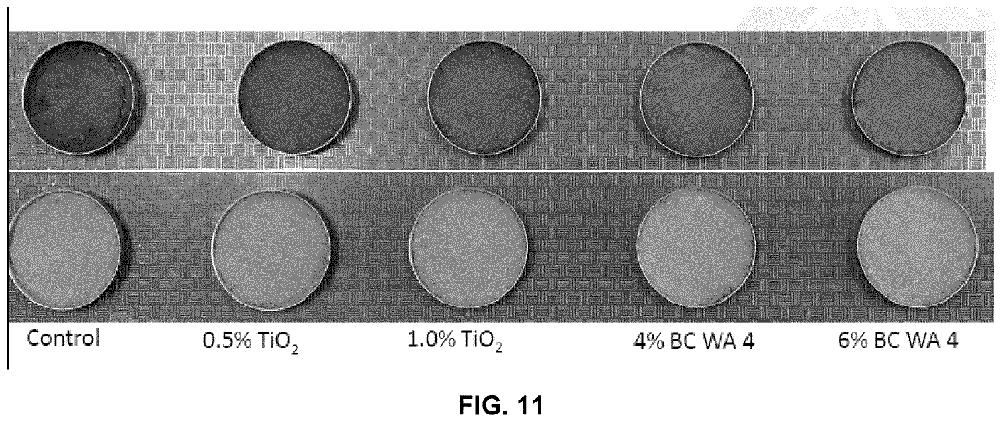

FIG. 12 is an image showing the whitening affect of BC WA 4 as compared to TiO₂ in toothpaste.

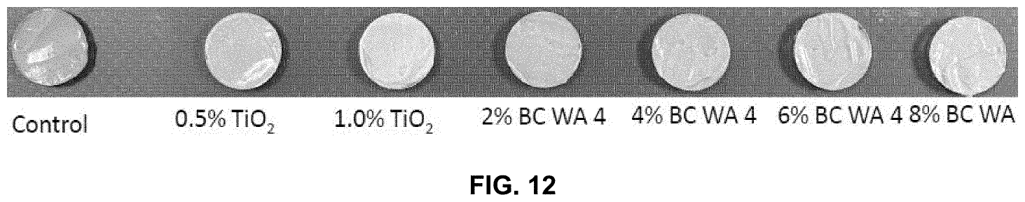

FIG. 13 is an image showing the whitening affect of BC WA 4 as compared to TiO₂ in laundry detergent.

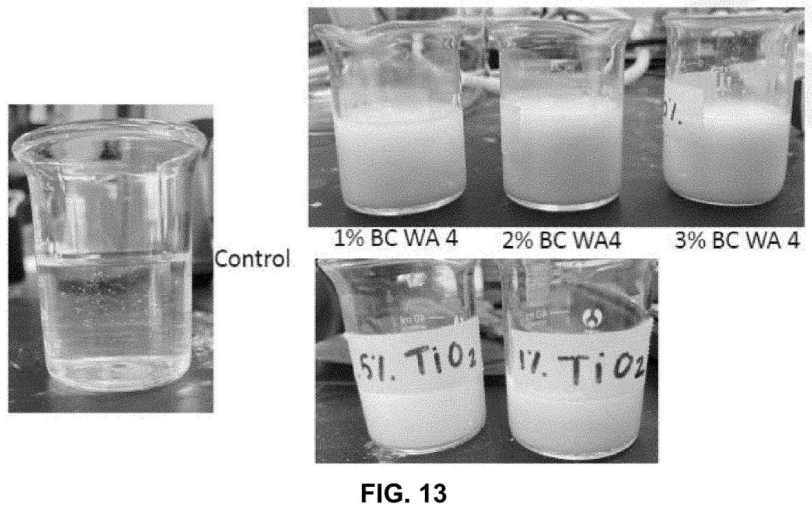

### DETAILED DESCRIPTION OF THE INVENTION

In one embodiment, the disclosure provides a dry powder for whitening a food, or pharmaceutical product comprising spray-dried particles, wherein:
(i) the spray-dried particles comprise microcapsules; and
(ii) the microcapsules comprise an inner core and a primary shell surrounding the inner core, wherein (a) the inner core comprises food grade CaCO₃ and/or zinc oxide (ZnO); and (b) the primary shell comprises food grade modified starch or gum Arabic.

In another embodiment, the disclosure provides a dry powder for whitening a food, or pharmaceutical product comprising spray-dried particles, wherein:
(i) the spray-dried particles comprise microcapsules; and
(ii) the microcapsules comprise an inner core and a primary shell surrounding the inner core, wherein (a) the inner core comprises food grade CaCO₃; and (b) the primary shell comprises food grade modified starch or gum Arabic.

In another embodiment, the disclosure provides a dry powder for whitening a food, or pharmaceutical product comprising spray-dried particles, wherein:
(i) the spray-dried particles comprise microcapsules; and
(ii) the microcapsules comprise an inner core and a primary shell surrounding the inner core, wherein (a) the inner core comprises food grade zinc oxide (ZnO); and (b) the primary shell comprises food grade modified starch or gum Arabic.

In another embodiment, the disclosure provides a dry powder for whitening a food, or pharmaceutical product comprising spray-dried particles, wherein:
(i) the spray-dried particles comprise microcapsules; and
(ii) the microcapsules comprise an inner core and a primary shell surrounding the inner core, wherein (a) the inner core comprises food grade CaCO₃ and zinc oxide (ZnO); and (b) the primary shell comprises food grade modified starch or gum Arabic.

In another embodiment, the disclosure provides a dry powder for whitening a food, or pharmaceutical product comprising spray-dried particles, wherein (i) the spray-dried particles comprise microcapsules.

In another embodiment, the disclosure provides a dry powder described above, wherein the redispersion diameter of the spray-dried particles is less than 20 μm.

In another embodiment, the disclosure provides a dry powder described above, wherein the redispersion diameter of the spray-dried particles is about 1 μm to about 15 μm.

In another embodiment, the disclosure provides a dry powder described above having an L* value above 90 in the CIELAB color space.

In another embodiment, the disclosure provides a dry powder described above having an a* value below 1.0 in the CIELAB color space.

In another embodiment, the disclosure provides a dry powder described above having a b* value below 9.0 in the CIELAB color space.

In another embodiment, the disclosure provides a dry powder described above that is stable.

In another embodiment, the disclosure provides a dry powder described above, wherein the food grade modified starch is N-LOK 1930, HICAP 100, THERMFLO, PURITY GUM ULTRA, CAPUSL IP, CAPSUL TA, CAPSUL 2730, NATIONAL 912, NATIONAL 1545, NATIONAL 4302, N-LOK, N-ZORBITM, N-ZORBIT 2144, N-DULGE CA1, N-CREAMER 115, COLFLO 67, PURITY CSC, CLEARGEL, CRISP COAT 764, or Gum Arabic. In another embodiment, the food grade modified starch is N-LOK 1930 or HICAP 100. In another embodiment, the food grade modified starch is N-LOK 1930. In another embodiment, the food grade shell material is gum Arabic.

In another embodiment, the disclosure provides a food comprising a sufficient amount of the dry powder described above to provide whitening to the food, e.g., the food is a beef, chicken, shrimp, or pork analog. In another embodiment, the food comprises about 0.01% to about 10% w/w of the dry powder, e.g., about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10% w/w of the dry powder.

In another embodiment, the disclosure provides a beverage comprising a sufficient amount of the dry powder described above to provide whitening to the beverage. In another embodiment, the beverage comprises about 0.01% to about 10% w/w of the dry powder, e.g., about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10% w/w of the dry powder.

In another embodiment, the disclosure provides a supplement comprising a sufficient amount of the dry powder described above to provide whitening to the supplement. In another embodiment, the supplement comprises about 0.01% to about 10% w/w of the dry powder, e.g., about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10% w/w of the dry powder.

In another embodiment, the disclosure provides a pharmaceutical product, e.g., a chewable tablet, comprising a sufficient amount of the dry powder described above to provide whitening to the product. In another embodiment, the pharmaceutical product comprises about 0.01% to about 10% w/w of the dry powder, e.g., about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10% w/w of the dry powder.

In another embodiment, the disclosure provides a cosmetic product, e.g., eye shadow, comprising a sufficient amount of the dry powder described above to provide whitening to the product. In another embodiment, the cosmetic product comprises about 0.01% to about 10% w/w of the dry powder, e.g., about 0.01%, about 0.1%, about 0.2%, about 0.3%, ab out 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10% w/w of the dry powder.

In another embodiment, the disclosure provides a personal care product, e.g., shampoo, toothpaste, or lip balm, comprising a sufficient amount of the dry powder described above to provide whitening to the product. In another embodiment, the personal care product comprises about 0.01% to about 10% w/w of the dry powder, e.g., about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10% w/w of the dry powder

Unless stated otherwise, the term “microcapsule” as used herein refers to single-core, multicore, or a mixture of single-core and multicore microcapsules.

Particle size can be measured using any typical equipment known in the art, for example, a Zetasizer Ultra, Malvern, Worcestershire, UK.

The microcapsules described in this section are collectively referred to as “Microcapsules of the Disclosure” (each individually referred to as a “Microcapsule of the Disclosure”).

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular

Measuring Color: The three values L*, a*, b* are used to characterize the principal color in the CIELAB model specified by the International Commission on Illumination (Commission Internationale d'Eclairage). It describes all the colors visible to the human eye and was created to serve as a device independent model to be used as a reference. L* denotes the luminosity and extends from 0 (black) to 100 (white). The value a* measures the red and green of the color: the colors tending toward green have a negative value while those tending toward the red have a positive a* value. The b* value measures the blue and the yellow of the color: colors tending toward the yellow have a positive b* value while those tending toward the blue have a negative b* value. The L*, a*, b* values are measured using a spectrum colorimeter.

As used herein, the singular forms “a,” “an,” and “the” include plural references unless the context clearly dictates otherwise. For example, the term “a compound” or “at least one compound” may include a plurality of compounds, including mixtures thereof. The terms “a”, “an,” “the,” “one or more,” and “at least one,” for example, can be used interchangeably herein.

As used herein, the term “about,” means plus or minus 10% of the reported numerical value.

Various embodiments of this disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range, such as from 1 to 6 should be considered to have specifically disclosed subranges such as from 1 to 2, from 1 to 3, from 1 to 4, from 1 to 5, from 2 to 3, from 2 to 4, from 2 to 5, from 2 to 6, from 3 to 4, from 3 to 5, from 3 to 6, etc., as well as individual numbers within that range, for example, 1, 2, 3, 4, 5, and 6. This applies regardless of the breadth of the range.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The terms “comprises,” “comprising,” “includes,” “including,” “having,” and their conjugates are interchangeable and mean “including but not limited to.” It is understood that wherever aspects are described herein with the language “comprising,” otherwise analogous aspects described in terms of “consisting of” and/or “consisting essentially of” are also provided.

The term “consisting of means” including and limited to.”

The term “consisting essentially of” means the specified material of a composition, or the specified steps of a method, and those additional materials or steps that do not materially affect the basic characteristics of the material or method.

The term “food grade” as used herein refers to material that is safe for human consumption.

A weight percent (wt. %) of a component is based on the total weight of the composition in which the component is included.

### EXAMPLES

### Example 1

### Preparation of CaCO₃-Loaded Microcapsules

Step 1. Weighed 180 g modified starch N-LOK1930 in 900 g deionized water at 50° C. under agitation (>300 rpm) until uniformly dissolved.

Step 2. Homogenized at 15 K rpm for 15 minutes, and then added 120 g CaCO₃ gradually.

Step 3. Slurried with average particle size<1 μm.

Step 4. Spray dried to give a powder, referred to herein as BC WA 4, with an L* value>92

As shown in the figures, 2-6% BC WA 4 works as well as or better than 1.0% TiO₂ regarding whitening effects in food, beverage, supplement, cosmetic, and personal care products.

### Example 2

### Preparation of ZnO-Loaded Microcapsules

1. Heated 112.50 g of water (not boiling) to about −80° C.
2. Added 37.50 g of Gum Arabic to the hot water and agitate until its redispersion is uniform (slurry).
3. Added 56.25 g of ZnO during the initial stages of emulsification. Emulsified/homogenized in total for 7 minutes @ 15K RPM (performed in a chilled reactor to reduce the localized temperature of the generator probe).
4. Spray dried the prepared emulsion with the following parameters:
a. Inlet Temperature: 160° C.
b. Blow Speed: 50 Hz
c. Feed Rate: 2 mL/min
d. Atomization: 0.22 mPa
e. Outlet temperature ˜95-100° C.
to provide a powder.

The above procedure was repeated at approximately 3× scale to provide a powder having the following color parameters: L*=94.47 (0.01); a*=−1.21 (0.01); b*=6.72 (0.02).

### Example 3

### Preparation of CaCO₃ and ZnO-Loaded Microcapsules

(1:1 ZnO: CaCO₃) w/20% N-Lok 1930 at 80% Solids by Straight Spray Dry
1. Dissolved 90 g of N-Lok 1930 into 450 g of DI water at 60 to 80° C. under stirring until fully redispersed
2. Gradually added 180 g of zinc oxide+180 g CaCO₃ into the above suspension
3. Homogenized the above suspension until the average particle size is around 1 micron (15000 RPM for 7 minutes)
4. Spray-dried the suspension to provide a powder.
5. Packed the powder with a sealer.

A powder produced by this procedure had the following color parameters: L*=94.51; a*=−0.96; b*=4.05.

Having now fully described the methods, microcapsules, and compositions herein, it will be understood by those of skill in the art that the same can be performed within a wide and equivalent range of conditions, formulations, and other parameters without affecting the scope of the methods, compounds, and compositions provided herein or any embodiment thereof. All patents, patent applications, and publications cited herein are fully incorporated by reference herein in their entirety.

1. A dry powder for whitening a food, or pharmaceutical or consumer product comprising spray-dried particles, wherein:
(i) the spray-dried particles comprise microcapsules; and
(ii) the microcapsules comprise an inner core and a primary shell surrounding the inner core, wherein (a) the inner core comprises food grade calcium carbonate and/or zinc oxide; and (b) the primary shell comprises food grade modified starch or gum Arabic.

2. The dry powder of claim 1, wherein the redispersion diameter of the spray-dried particles is about 1 μm to about 15 μm.

3. The dry powder of claim 1 having an L* value above 90 in the CIELAB color space.

4. The dry powder of claim 1 having an a* value below 1.0 in the CIELAB color space.

5. The dry powder of claim 1 having a b* value below 9.0 in the CIELAB color space.

6. The dry powder of claim 1 that is stable.

7. The dry powder of claim 1, wherein the food grade modified starch is N-LOK 1930, HICAP 100, THERMFLO, PURITY GUM ULTRA, CAPUSL IP, CAPSUL TA, CAPSUL 2730, NATIONAL 912, NATIONAL 1545, NATIONAL 4302, N-LOK, N-ZORBITM, N-ZORBIT 2144, N-DULGE CA1, N-CREAMER 115, COLFLO 67, PURITY CSC, CLEARGEL, CRISP COAT 764, or Gum Arabic, or a combination thereof.

8. The dry powder of claim 7, wherein the food grade modified starch is N-LOK 1930 or HICAP 100.

9. The dry powder of claim 8, wherein the food grade modified starch is N-LOK 1930.

10. The dry powder of claim 1, wherein the primary shell comprises gum Arabic.

11. A food comprising a sufficient amount of the dry powder of claim 1 to provide whitening to the food.

12. The food of claim 11, wherein the food is a beef, chicken, shrimp, or pork analog.

13. The food of claim 11, wherein the food comprises about 0.01% to about 10% w/w of the dry powder.

14. A beverage comprising a sufficient amount of the dry powder of claim 1 to provide whitening to the beverage.

15. The beverage of claim 14, wherein the beverage comprises about 0.01% to about 10% w/w of the dry powder.

16. A supplement comprising a sufficient amount of the dry powder of claim 1 to provide whitening to the supplement.

17. The supplement of claim 16, wherein the supplement comprises about 0.01% to about 10% w/w of the dry powder.

18. A pharmaceutical product comprising a sufficient amount of the dry powder of claim 1 to provide whitening to the product.

19. The pharmaceutical product of claim 18 that is a chewable tablet, gelatin or vegan capsule, or thin film.

20. The pharmaceutical product of claim 19, wherein the product comprises about 0.01% to about 10% w/w of the dry powder.

21. A cosmetic product comprising a sufficient amount of the dry powder of claim 1 to provide whitening to the product.

22. The cosmetic product of claim 21, wherein the product comprises about 0.01% to about 10% w/w of the dry powder.

23. A personal care product comprising a sufficient amount of the dry powder of any one of claim 1 to provide whitening to the product.

24. The personal care product of claim 23, wherein the product comprises about 0.01% to about 10% w/w of the dry powder.
